(19) United States
(12) Patent Application Publication
(10) Pub. No.: US 2021/0030292 A1
(43) Pub. Date: Feb. 4, 2021

(54) SOFT MULTI-SEGMENTED, FUNCTIONALIZED BODIES MADE FROM POLYMERS

(71) Applicants: Heraeus Deutschland GmbH & Co. KG, Hanau (DE); Heraeus Medical Components LLC, St. Paul, MN (US)

(72) Inventors: Robert Dittmer, Hanau (DE); Leonard Stoica, Hanau (DE); Jeffrey Hendricks, St. Paul, MN (US)

(73) Assignees: Heraeus Deutschland GmbH & Co. KG, Hanau (DE); Heraeus Medical Components LLC, St. Paul, MN (US)

(21) Appl. No.: 17/031,111

(22) Filed: Sep. 24, 2020

Related U.S. Application Data
(60) Provisional application No. 62/881,628, filed on Aug. 1, 2019.

(51) Int. Cl. A61B 5/04; A61B 5/00; A61N 1/04; A61N 1/36; B33Y 10/00; B33Y 80/00; B33Y 70/00; B29C 64/112

(52) U.S. Cl. CPC A61B 5/04; A61B 5/681; A61B 5/6804; A61B 5/6803; A61N 1/0456; A61N 1/0484; A61B 5/0492; B33Y 10/00; B33Y 80/00; B33Y 70/00; B29C 64/112; A61B 5/6815; A61N 1/36014

(57) ABSTRACT

One aspect relates to a body, including at least one first polymer segment having a porosity p1 and an electrical conductivity c1, and at least one further polymer segment being in seamless contact with the at least one first polymer segment and having a porosity p2≠p1 and an electrical conductivity c2≠c1. One aspect also relates to a process for the preparation of a body, to a body obtainable by such a process, to the use of a body for electrophysical measurements or as a biosensor and to a therapeutic current delivery or current receiving system comprising the body.

### CROSS-REFERENCED TO RELATED APPLICATION

This Non-Provisional patent application claims the benefit of the filing date of U.S. Provisional Patent Application Ser. No. 62/881,628, filed Aug. 1, 2019, which is incorporated herein by reference.

### TECHNICAL FIELD

One aspect relates to a body, comprising at least one first polymer segment and at least one further polymer segment, wherein these segments are in seamless contact and differ with respect to their porosity and their electrical conductivity. One aspect also relates to a process for the preparation of a body, to a body obtainable by such a process, to the use of a body for electrophysical measurements and to a therapeutic current delivery or current receiving system comprising the body.

### BACKGROUND

Skin surface electrodes are important components in many medical diagnostic systems including, for example, electrocardiography (ECG), electromyography (EMG), and electroencephalography (EEG). In these systems, the electrode plays a critical role as a transducer converting physiological variables, such as those of the heart, muscles and brain, respectively, in ECG, EMG and EEG, to electrical potentials (sometime referred to as biopotentials). The measured potentials are then amplified and processed by an instrument, such as an electronic measuring circuit and a computer. The workings of skin surface electrodes are dependent on a multitude of mechanisms, such as electrode material, the electrolyte applied to the electrode, body location, and skin properties. The electrode must convert a physiological ionic current into an electronic current receivable and readable by the associated instrumentation.

State of the art in the field of current-carrying and/or dissipating body electrodes optimized for short-term applications are systems that are based on aqueous gel electrolytes, such as those disclosed in U.S. Pat. No. 9,050,451 B1. As an alternative for long-term applications electrodes are known in the prior art that are based on polymers comprising carbon, such as the graphite loaded polymers disclosed in U.S. Pat. No. 7,136,691 B2, or electrodes that are based on conductive liquid elastomers filled with metallic particles, such as the rubber compositions disclosed in DE 10 2008 050 932 A1. It is also known in the prior art that doped thiophenes such as PEDOT/PSS can be used as conductive materials in body electrodes. Leleux et al. (Adv. Healthcare Mater, 2014, 3, pages 1377-1380) disclose ionic liquid gel-assisted electrodes for long-term cutaneous recordings, in which an ionic liquid gel was incorporated onto electrodes made of Au and PEDOT/PSS. These electrodes were deposited on a thin film of parylene C to render them conformable to the skin.

However, the electrodes known from the prior art, in which conductive materials such as PEDOT/PSS are simply coated onto a non-conductive support, are disadvantageous in several ways. First of all, for certain applications multi-channel electrodes are required in which two or more electrodes have to be accommodated in close neighborhood to each other, which by means of the prior art approaches for forming electrodes are often difficult to produce. Furthermore, skin surface electrodes often also must be characterized by a certain softness of the material that allows an optimized fit of the electrode, for example, in the ear of a patient. Also, if the electrode pathway is applied onto the surface of a nonconductive support, it is difficult to realize a sufficient robustness of the electrode, particularly in case of a multi-channel electrode.

### SUMMARY

One embodiment was based on the object of overcoming the disadvantages resulting from the prior art in connection with skin surface electrodes.

In particular, one embodiment is based on the object of providing a body that can, for example, be used as a skin surface electrode, and which combines the advantages of high elasticity and high robustness.

A contribution to at least one of the above objects is given by the independent claims. The dependent claims provide preferred embodiments, which also serve solving at least one of the above mentioned objects.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 a cross-sectional scheme of the body according to one embodiment;

FIG. 2A a side view of the body according to one embodiment in the design of an in-ear multiple electrode;

FIG. 2B the in-ear multiple electrode shown in FIG. 2A) in a top view;

FIG. 3 a scheme showing the deposition of droplets onto the surface of a substrate in a 3D-printing process according to one embodiment;

FIG. 4 two further schemes showing the deposition of droplets onto the surface of a substrate in a 3D-printing process according to one embodiment, in which three successive sheet layers are shown;

FIG. 5 the formation of the body shown in FIG. 1 by means of the process according to one embodiment;

FIG. 6 a scheme showing the process step of impregnating certain segments of the body according to the embodiment shown in FIG. 1 with PEDOT/PSS;

FIG. 7 the formation of the body shown in FIG. 2a by means of the process according to one embodiment;

FIG. 8A a scheme showing the process step of introducing PEDOT in certain segments of the body according to the embodiment shown in FIG. 2A;

FIG. 8B a further scheme showing the process step of introducing PEDOT in certain segments of the body according to the embodiment shown in FIG. 2A.

### DETAILED DESCRIPTION

In the following Detailed Description, reference is made to the accompanying drawings, which form a part hereof, and in which is illustrated by way of illustration specific embodiments in which one embodiments may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present embodiments. The following detailed description, therefore, is not to be taken in a limiting sense, and the scope of the present embodiments are defined by the appended claims.

It is to be understood that the features of the various exemplary embodiments described herein may be combined with each other, unless specifically noted otherwise.

A contribution to the solution of at least one of the above objects is made by an embodiment 1 of a body 1, in one embodiment a polymer body 1, comprising
i) at least one first polymer segment having a porosity p1 and an electrical conductivity c1,
   and
ii) at least one further polymer segment being in seamless contact with the at least one first polymer segment and having a porosity p2≠p1 and an electrical conductivity c2≠c1.

In an embodiment 2 of the body 1 the body is designed according to its embodiment 1, wherein
p1>p2, and
c1>c2.
   In this context, it is particularly preferred that
p1>1.1×p2,
preferably p1>1.2×p2,
more preferably p1>1.5×p2,
even more preferably p1>2.0×p2,
even more preferably p1>5.0×p2,
even more preferably p1>10.0×p2, and
most preferably p1>100×p2,
and
c1>10×c2,
preferably c1>100×c2,
more preferably c1>500×c2,
even more preferably c1>1,000×c2,
even more preferably c1>10,000×c2,
even more preferably c1>100,000×c2, and
most preferably c1>1,000,000×c2.

In an embodiment 3 of the body 1 the body is designed according to its embodiment 2, wherein the further segment has a porosity p2 of less than 50%, more preferably of less than 20%, even more preferably of less than 5% and most preferably of less than 1% and wherein the first polymer segment has a porosity p1 of at least 10%, preferably of at least 25%, even more preferably of at least 50% and most preferably of at least 75%.

In an embodiment 4 of the body 1 the body is designed according to its embodiment 2 or 3, wherein the first polymer segment has an electrical conductivity c1 of at least 0.001 S/cm, preferably of at least 0.01 S/cm, more preferably of at least 0.1 S/cm and most preferably of at least 1 S/cm and wherein the further polymer segment has an electrical conductivity c2 of less than 1×10⁻⁶ S/cm, preferably of less than 1×10⁻⁷ S/cm, more preferably of less than 1×10⁻⁸ S/cm and most preferably of less than 1×10⁻⁹ S/cm.

In an embodiment 5 of the body 1 the body is designed according to its embodiment 1, wherein p1>p2, and c2>c1.

In this context, it is particularly preferred that
p1>1.1×p2,
preferably p1>1.2×p2,
more preferably p1>1.5×p2,
even more preferably p1>2.0×p2,
even more preferably p1>5.0×p2,
even more preferably p1>10.0×p2, and
most preferably p1>100×p2,
and
c2>10×c1,
preferably c2>100×c1,
more preferably c2>500×c1,
even more preferably c2>1,000×c1,
even more preferably c2>10,000×c1,
even more preferably c2>100,000×c1, and
most preferably c2>1,000,000×c1.

In an embodiment 6 of the body 1 the body is designed according to its embodiment 5, wherein the further segment has a porosity p2 of less than 50%, more preferably of less than 20%, even more preferably of less than 5% and most preferably of less than 1% and wherein the first polymer segment has a porosity p1 of at least 10%, preferably of at least 25%, even more preferably of at least 50% and most preferably of at least 75%.

In an embodiment 7 of the body 1 the body is designed according to its embodiment 5 or 6, wherein the first polymer segment has an electrical conductivity c1 of less than 1×10⁻⁶ S/cm, preferably of less than 1×10⁻⁷ S/cm, more preferably of less than 1×10'S/cm and most preferably of less than 1×10⁻⁹ S/cm and that the further polymer segment has an electrical conductivity c2 of at least 0.001 S/cm, preferably of at least 0.01 S/cm, more preferably of at least 0.1 S/cm and most preferably of at least 1 S/cm.

In an embodiment 8 of the body 1 the body is designed according to anyone of its embodiments 1 to 7, wherein the first polymer segment has compression modulus m1 and wherein the further polymer segment has a compression modulus m2≠m1.

In an embodiment 9 of the body 1 the body is designed according to its embodiment 8, wherein the first polymer segment has a compression modulus m2 of at least 0.01 MPa, preferably of at least 1 MPa, more preferably of at least 50 MPa and most preferably of at least 1000 MPa and wherein the further polymer segment has a compression modulus m1 of less than 5000 MPa, preferably of less than 1000 MPa, more preferably of less than 500 MPa and most preferably of less than 100 MPa.

In an embodiment 10 of the body 1 the body is designed according to its embodiment 8, wherein the first polymer segment has a compression modulus m1 of less than 5000 MPa, preferably of less than 1000 MPa, more preferably of less than 500 MPa and most preferably of less than 100 MPa and wherein the further polymer segment has a compression modulus m2 of at least 0.01 MPa, preferably of at least 1 MPa, more preferably of at least 50 MPa and most preferably of at least 1000 MPa.

In an embodiment 11 of the body 1 the body is designed according to anyone of its embodiments 1 to 10, wherein at least one segment selected from the first polymer segment and the further polymer segment includes, preferably is made from, a conductive material that includes a non-conductive polymer matrix and a conductive component dispersed therein.

In an embodiment 12 of the body 1 the body is designed according to its embodiment 11, wherein the non-conductive polymer matrix includes, in one embodiment is made from, a thermoplastic polymer, a silicone or a mixture thereof.

In an embodiment 13 of the body 1 the body is designed according to its embodiment 12, wherein the thermoplastic polymer is selected from the group consisting of acrylate or methylmethacrylate polymers or copolymers, acrylonitrile-butadiene-styrene polymers (ABS), polyvinylidene fluoride polymers, polyurethane polymers, polyolefin polymers, polyester polymers, polyamide polymers, polyimide polymers, styrene-ethylene-butylene styrene (SEBS) polymers, polyether ether ketone (PEEK) polymers, polyphenylene sulfide (PPS) polymers, polysulfone polymers, ethylene-vinyl acetate (EVA) polymers, polyethalene-naphthalate (PEN) polymers, aromatic polycarbonate polymers and mixtures of two or more of these polymers.

In an embodiment 14 of the body 1 the body is designed according to anyone of its embodiments 11 to 13, wherein the conductive component is selected from the group consisting of metal particles, carbon particles or a mixture thereof.

In an embodiment 15 of the body 1 the body is designed according to its embodiment 14, wherein the conductive component is selected from the group consisting of carbon nanofibers, carbon nanotubes, fullerenes, synthetic porous carbon powders like Porocarb® (Heraeus, Deutschland), carbon black powder, graphene nanoplatelets, particles based on metals such as silver, copper, nickel or aluminum, particles based on precious metals such as platinum, gold, iridium and alloys thereof, particles based on refractory metals such as titanium, zirconium, niobium, tantalum and alloys thereof, or mixtures of two or more of these conductive components.

In an embodiment 16 of the body 1 the body is designed according to its embodiment 10, wherein the conductive component includes a conductive polymer.

In an embodiment 17 of the body 1 the body is designed according to its embodiment 16, wherein the conductive polymer includes a polythiophene.

In an embodiment 18 of the body 1 the body is designed according to its embodiment 17, wherein the polythiophene is a cationic polythiophene that is present in the form of a polythiophene/polyanion-complex.

In an embodiment 19 of the body 1 the body is designed according to its embodiment 18, wherein the polythiophene/polyanion-complex is a PEDOT/PSS-complex.

In an embodiment 20 of the body 1 the body is designed according to anyone of its embodiments 1 to 10, wherein at least one segment selected from the first polymer segment and the further polymer segment includes, in one embodiment is made from a non-conductive material.

In an embodiment 21 of the body 1 the body is designed according to its embodiment 20, wherein the non-conductive material includes, in one embodiment is made from or consists of, a non-conductive polymer as defined in embodiments 12 and 13.

In an embodiment 22 of the body 1 the body is designed according to anyone of its embodiments 1 to 21, wherein the first polymer segment includes, in one embodiment is made from, a conductive material as defined in anyone of embodiments 11 to 19 and wherein the further polymer segment includes, in one embodiment is made from, a non-conductive polymer as defined in embodiment 21.

In an embodiment 23 of the body 1 the body is designed according to anyone of its embodiments 1 to 21, wherein the first polymer segment includes, in one embodiment is made from, a non-conductive polymer as defined in embodiment 21 and wherein the further polymer segment includes, in one embodiment is made from, a conductive material as defined in anyone of embodiments 11 to 19.

In an embodiment 24 of the body 1 the body is designed according to anyone of its embodiments 1 to 23, wherein the first polymer segment and the further polymer segment include, in one embodiment are made from, the same non-conductive polymer, these polymers differing with respect to their porosity, wherein the first polymer segment further includes a conductive component as defined in embodiments 14 or 15, a conductive polymer as defined in anyone of embodiments 16 to 19 or a mixture thereof, wherein it is preferred that the conductive component is dispersed in a matrix of the non-conductive polymer, is coated onto at least a part of the outer surface of the first segment or is dispersed in a matrix of the non-conductive polymer and is coated onto at least a part of the outer surface of the first segment.

In an embodiment 25 of the body 1 the body is designed according to anyone of its embodiments 1 to 23, wherein the first polymer segment and the further polymer segment include, in one embodiment are made from, the same non-conductive polymer, these polymers differing with respect to their porosity, wherein the further polymer segment further includes a conductive component as defined in embodiments 14 or 15, a conductive polymer as defined in anyone of embodiments 16 to 19 or a mixture thereof, wherein it is preferred that the conductive component is dispersed in a matrix of the non-conductive polymer, is coated onto at least a part of the outer surface of the further segment or is dispersed in a matrix of the non-conductive polymer and is coated onto at least a part of the outer surface of the further segment.

In an embodiment 26 of the body 1 the body is designed according to anyone of its embodiments 1 to 25, wherein the body has been prepared by means of a 3D-printing process, a molding/overmolding process, a co-extrusion process, a casting process or a combination of these techniques, in one embodiment by means of a 3D-printing process.

In an embodiment 27 of the body 1 the body is designed according to its embodiment 26, wherein the 3D-printing process is an additive layer manufacturing (ALM) process.

In an embodiment 28 of the body 1 the body is designed according to its embodiment 26 or 27, wherein the 3D-printing process is a fused filament fabrication process, a process comprising the hot extrusion of rods, a process comprising the cold extrusion of rots or a process comprising the hot extrusion of pellets, the pellets, filament and rods in one embodiment comprising a non-conductive or conductive material as defined in the previous embodiments.

In an embodiment 29 of the body 1 the body is designed according to anyone of its embodiments 26 to 28, wherein the difference in the porosity of the first polymer segment and the further polymer segment is a consequence of a difference in the fill factor that has been selected when printing these segments.

In an embodiment 30 of the body 1 the body is designed according to anyone of its embodiments 1 to 29, wherein the body is an electrode, in one embodiment a skin electrode for detecting bioelectric signals, for example by means of EMG or EEG, more in one embodiment an in-ear multiple electrode.

In an embodiment 31 of the body 1 the body is designed according to its embodiment 30, wherein the further segment forms the electrically conductive part of the electrode and the first segment forms the non-conductive base in which the electrically conductive part of the electrode is embedded in, on the surface of which the electrically conductive part of the electrode is located or in which the electrically conductive part of the electrode is embedded in and on the surface of which the electrically conductive part of the electrode is located.

In an embodiment 32 of the body 1 the body is designed according to its embodiment 31, wherein the further segment forms the electrically conductive part of the electrode and the first segment forms the non-conductive base in which the electrically conductive part of the electrode is embedded in and on the surface of which the electrically conductive part of the electrode is located, wherein the electrically conductive part that is located on the surface of the base is in electrical contact with the electrically conductive part of the electrode that is embedded in the base, the latter one forming an pathway by means of which the electrically conductive part that is located on the surface can be connected with a measuring device.

In an embodiment 33 of the body 1 the body is designed according to its embodiments 31 and 32, wherein the electrically conductive parts of the electrode on the surface of the base are the skin electrodes.

In an embodiment 34 of the body 1 the body is designed according to anyone of its embodiments 1 to 29, wherein the body is a biosensor or is part of a biosensor.

In an embodiment 35 of the body 1 the body is designed according to anyone of its embodiments 1 to 29, wherein the body is included in a device selected from the group consisting of, or is a device selected from the group consisting of a body-warn device having electrically sensing functionality, such as an EEG-electrode, an ECG-electrode, an EMG-electrode, an ERG-electrode, an EOG-electrode, an VEP-electrode or an AEP-electrode, a device having electrical stimulation functionality, such as a TENS-electrode, an EMS-electrode, a tVNS-electrode, a CES-electrode or a tDCS-electrode, or a body-worn device such as a headphone, glasses, a headband, a chest-strap, an armband, a patch, a garment or wrist-worn device.

A contribution to the solution of at least one of the above objects is made by an embodiment 1 of a process 1 comprising as process steps
I) preparing, by means of 3D-printing, in one embodiment by 3D-printing in an additive layer manufacturing (ALM) process, at least one first polymer segment having a porosity p1, in one embodiment by using a first fill factor f1, and
II) preparing, by means of 3D-printing, in one embodiment by 3D-printing in an additive layer manufacturing (ALM) process, at least one further polymer segment being in seamless contact with the at least one first polymer segment having a porosity p2≠p1, in one embodiment by using a second fill factor f2≠f1;
III) optionally bringing into contact a conductive material with a segment selected from the first polymer segment and the further polymer segment.

The difference in the porosity between the first and the further polymer segment can not only be controlled by adjusting the fill factor, but also by other measures such as the use of blowing agents.

In an embodiment 2 of the process 1 the process is designed according to its embodiment 1, wherein the 3D-printing process is an additive layer manufacturing (ALM) process.

In an embodiment 3 of the process 1 the process is designed according to its embodiments 1 or 2, wherein the 3D-printing process is a fused filament fabrication process, a process comprising the hot extrusion of rods, a process comprising the cold extrusion of rots or a process comprising the hot extrusion of pellets, the pellets, filament and rods in one embodiment comprising a non-conductive or conductive material as defined in the previous embodiments.

In an embodiment 4 of the process 1 the process is designed according to anyone of its embodiments 1 to 3, wherein the body is prepared in layers by depositing droplets of a molten polymer onto a surface of a substrate or onto a surface of a previously applied layer, in one embodiment onto the outer surface of a previously applied layer, wherein in the preparation of at least some of the layers a first area and a second area are formed which differ with respect to the density in which the droplets are deposited. The first and the second area in the two-dimensional form result in the first and the second segment in the three-dimensional form of the body if the individual layers are printed on top of each other in the 3D printing process.

In an embodiment 5 of the process 1 the process is designed according to anyone of its embodiments 1 to 4, wherein f1 is in the range from 5 to 100%, in one embodiment in the range from 10 to 90%, in one embodiment in the range from 15 to 80% and in one embodiment in the range from 20 to 70%.

In an embodiment 6 of the process 1 the process is designed according to anyone of its embodiments 1 to 5, wherein f2 is in the range from 5 to 100%, preferably in the range from 10 to 90%, more preferably in the range from 15 to 80% and most preferably in the range from 20 to 70%.

In an embodiment 7 of the process 1 the process is designed according to anyone of its embodiments 1 to 6, wherein at least one segment selected from the first polymer segment and the further polymer segment is made from a conductive material that includes a non-conductive polymer and a conductive component dispersed therein.

In an embodiment 8 of the process 1 the process is designed according to its embodiment 7, wherein the non-conductive polymer is a thermoplastic polymer, a silicone or a mixture thereof.

In an embodiment 9 of the process 1 the process is designed according to its embodiment 8, wherein the thermoplastic polymer is selected from the group consisting of acrylate or methylmethacrylate polymers or copolymers, acrylonitrile-butadiene-styrene polymers (ABS), polyvinylidene fluoride polymers, polyurethane polymers, polyolefin polymers, polyester polymers, polyamide polymers, polyimide polymers, styrene-ethylene-butylene styrene (SEBS) polymers, polyether ether ketone (PEEK) polymers, polyphenylene sulfide (PPS) polymers, polysulfone polymers, ethylene-vinyl acetate (EVA) polymers, polyethalene-naphthalate (PEN) polymers, aromatic polycarbonate polymers and mixtures of two or more of these polymers.

In an embodiment 10 of the process 1 the process is designed according to its embodiment 8 or 9, wherein the conductive component is a metal particle, a carbon particles or a mixture thereof.

In an embodiment 11 of the process 1 the process is designed according to its embodiment 10, wherein the conductive component is selected from the group consisting of carbon nanofibers, carbon nanotubes, fullerenes, synthetic porous carbon powders like Porocarb® (Heraeus, Deutschland), carbon black powder, graphene nanoplatelets, particles based on metals such as silver, copper, nickel or aluminum, particles based on precious metals such as platinum, gold, iridium and alloys thereof, particles based on refractory metals such as titanium, zirconium, niobium, tantalum and alloys thereof, or mixtures of two or more of these conductive component.

In an embodiment 12 of the process 1 the process is designed according to anyone of its embodiments 1 to 11, wherein at least one segment selected from the first polymer segment and the further polymer segment is made from a non-conductive material.

In an embodiment 13 of the process 1 the process is designed according to anyone of its embodiments 1 to 12, wherein the non-conductive material is a non-conductive polymer as defined in embodiments 8 and 9.

In an embodiment 14 of the process 1 the process is designed according to anyone of its embodiments 1 to 13, wherein in process step I) the first polymer segment is made from a conductive material as defined in anyone of embodiments 7 to 11 and wherein the further polymer segment is made from a non-conductive polymer as defined in embodiments 8 and 9.

In an embodiment 15 of the process 1 the process is designed according to anyone of its embodiments 1 to 13, wherein the first polymer segment is made from a non-conductive polymer as defined in embodiments 8 and 9 and wherein the further polymer segment is made from a conductive material as defined in anyone of embodiments 7 to 11.

In an embodiment 16 of the process 1 the process is designed according to anyone of its embodiments 1 to 15, wherein the conductive material that in process step III) is brought into contact a with a segment selected from the first polymer segment and the further polymer segment is a conductive component as defined in claim 10 or 11, a conductive polymer or a mixture of these conductive materials.

In an embodiment 17 of the process 1 the process is designed according to its embodiment 16, wherein the conductive polymer includes a polythiophene.

In an embodiment 18 of the process 1 the process is designed according to its embodiment 17, wherein the polythiophene is a cationic polythiophene that is present in the form of a polythiophene/polyanion-complex.

In an embodiment 19 of the process 1 the process is designed according to its embodiment 18, wherein the polythiophene/polyanion-complex is a PEDOT/PSS-complex.

In an embodiment 20 of the process 1 the process is designed according to anyone of its embodiments 1 to 19, wherein at least one segment selected from the first polymer segment and the further polymer segment includes void spaces and wherein in process step III) a conductive material is introduced into at least some of the void spaces or wherein in process step III) at least a part of the surface of at least one segment selected from the first polymer segment and the further polymer segment is coated with a conductive material.

In an embodiment 21 of the process 1 the process is designed according to its embodiment 20, wherein the conductive material is a conductive component as defined in embodiments 10 or 11 or a conductive polymer as defined in anyone of embodiments 17 to 19.

In an embodiment 22 of the process 1 the process is designed according to its embodiment 20 or 21, wherein in process steps I) and II) the first polymer segment and the further polymer segment are made from the same non-conductive polymer, these polymers differing with respect to their porosity, and wherein in process step III) a conductive component as defined in embodiments 10 or 11, a conductive polymer as defined in anyone of embodiments 17 to 19 or a mixture thereof is brought into contact with the first polymer segment.

In an embodiment 23 of the process 1 the process is designed according to its embodiment 20 or 21, wherein in process steps I) and II) the first polymer segment and the further polymer segment are made from the same non-conductive polymer, these polymers differing with respect to their porosity, and wherein in process step III) a conductive component as defined in embodiments 10 or 11, a conductive polymer as defined in anyone of embodiments 17 to 19 or a mixture thereof is brought into contact with the further polymer segment.

In an embodiment 24 of the process 1 the process is designed according to anyone of its embodiments 20 to 23, wherein in process step III) the conductive polymer is introduced into the void spaces by introducing a solution comprising monomers of the conductive polymer and forming the conductive polymer within the void spaces by electrodeposition, by introducing a solution or dispersion that includes a solvent and the conductive polymer into the void spaces and subsequently removing at least a part of the solvent, or by a combination of these techniques.

A contribution to the solution of at least one of the above objects is made by an embodiment 1 of a body 2, obtainable by the process 1 according to anyone of embodiments 1 to 24.

In an embodiment 2 of the body 2 the body is designed according to its embodiment 1, wherein the body is an electrode, in one embodiment a skin electrode for detecting bioelectric signals, for example by means of EEG, EMG or EEG, more in one embodiment an in-ear multiple electrode.

In an embodiment 3 of the body 2 the body is designed according to its embodiment 1, wherein the body is a biosensor or is part of a biosensor.

In an embodiment 4 of the body 2 the body is designed according its embodiment 1, wherein the body is included in a device selected from the group consisting of, or is a device selected from the group consisting of a body-warn device having electrically sensing functionality, such as an EEG-electrode, an ECG-electrode, an EMG-electrode, an ERG-electrode, an EOG-electrode, an VEP-electrode or an AEP-electrode, a device having electrical stimulation functionality, such as a TENS-electrode, an EMS-electrode, a tVNS-electrode, a CES-electrode or a tDCS-electrode, or a body-worn device such as a headphone, glasses, a headband, a chest-strap, an armband, a patch, a garment or wrist-worn device.

A contribution to the solution of at least one of the above objects is also made by the use of the body 1 according to anyone of its embodiments 1 to 29 or by the use of the body 2 according to anyone of its embodiments 1 to 3 for electrophysical measurements, wherein at least a part of at least one segment selected from the first polymer segment and the further polymer segment comes into direct contact with the skin.

A contribution to the solution of at least one of the above objects is made by a therapeutic current delivery or current receiving system comprising:
a) a therapeutic current delivery and/or current receiving device;
b) at least one cable connector connected to the therapeutic current delivery or current receiving device;
c) at least one body 1 according to anyone of its embodiments 1 to 29 or at least one body 2 according to its embodiments 1 or 2 connected to the at least one cable connector.

### The Body According to One Embodiment

The body according to one embodiment is a polymer body (i.e. a body which includes at least 10 wt.-%, more preferably at least 25 wt.-% and most preferably at least 50 wt.-% of a polymer, preferably a thermoplastic polymer, based on the total weight of the body.

The body is characterized in that it includes at least one first polymer segment and at least one further polymer segment, wherein these segments differ with respect to their porosity and their electrical conductivity and wherein these two segments are in seamless contact with each other. As used herein, the term “in seamless contact” is in one embodiment understood in such a way that the material of the first segment and the material of the further segment flow into each other in the border area between these two segments (i. e. they form a coherent material). The first and the further segment are in one embodiment not glued, welded of soldered to one another. Such a seamless contact is usually achieved when producing the first and further segment by means of 3D-printing in which these regions are produced by dropwise deposition of a molten thermoplastic material. In such a process drops which have been deposited to form the first segment come into contact with drops which have been deposited to form the further segment, wherein in the border region of the different segments the drops at least partially “fuse” and/or “entangle” with one another before they solidify so that a seamless transition between the segments is obtained.

The term “segment” as used herein refers to any three-dimensional structure that may be a part of a complex object that has been obtained in a 3D-printing process. The object may thus have any dimensional shape such as a rod, a plate, a sphere, a pyramid, a cube, or any part-section or combination of these shapes.

Furthermore, a body in which the first and the segment differ with respect to both, their porosity p and their conductivity c, also includes embodiments in which one of these segments is not porous at all (i. e. is built from a completely solid mass that in one embodiment does not include any voids at all) as well as embodiments in which one of the segments does not show any practically useful electrical conductivity at all (i. e. is built from an insulating material).

### The Conductive Polymer

The conductive polymer that may be included in the body according to one embodiment or that in process step III) of the process according to one embodiment may be brought into contact with a segment selected from the first polymer segment and the further polymer segment can be a polymer selected from the group consisting of an optionally substituted polythiophene, an optionally polyaniline and an optionally substituted polypyrrole, wherein optionally substituted polythiophenes are particularly preferred as the conductive polymer.

Preferred polythiophenes are those having the general formula

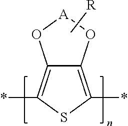

in which
A represents an optionally substituted C₁-C₅-alkylene radical,
R represents a linear or branched, optionally substituted C₁-C₁₈-alkyl radical, an optionally substituted C₅-C₁₂-cycloalkyl radical, an optionally substituted C₆-C₁₄-aryl radical, an optionally substituted C₇-C₁₈-aralkyl radical, an optionally substituted C₁-C₄-hydroxyalkyl radical or a hydroxyl radical, wherein 0 to 8 radicals R can be bonded to A and, in the case of more than one radical, can be identical or different.

The polythiophenes in one embodiment in each case carry H on the end groups.

In the context of one embodiment, C₁-C₅-alkylene radicals A are preferably methylene, ethylene, n-propylene, n-butylene or n-pentylene. C₁-C₁₈-alkyl R preferably represent linear or branched C₁-C₁₈-alkyl radicals, such as methyl, ethyl, n- or iso-propyl, n-, iso-, sec- or tert-butyl, n-pentyl, 1-methylbutyl, 2-methylbutyl, 3-methylbutyl, 1-ethylpropyl, 1,1-dimethylpropyl, 1,2-dimethylpropyl, 2,2-dimethylpropyl, n-hexyl, n-heptyl, n-octyl, 2-ethylhexyl, n-nonyl, n-decyl, n-undecyl, n-dodecyl, n-tridecyl, n-tetradecyl, n-hexadecyl or n-octadecyl, C₅-C₁₂-cycloalkyl radicals R represent, for example, cyclopentyl, cyclohexyl, cycloheptyl, cyclooctyl, cyclononyl or cyclodecyl, C₅-C₁₄-aryl radicals R represent, for example, phenyl or naphthyl, and C₇-C₁₈-aralkyl radicals R represent, for example, benzyl, o-, m-, p-tolyl, 2,3-, 2,4-, 2,5-, 2,6-, 3,4-, 3,5-xylyl or mesityl. The preceding list serves to illustrate one embodiment by way of example and is not to be considered conclusive.

In the context of one embodiment, numerous organic groups are possible as optionally further substituents of the radicals A and/or of the radicals R, for example alkyl, cycloalkyl, aryl, aralkyl, alkoxy, halogen, ether, thioether, disulphide, sulphoxide, sulphone, sulphonate, amino, aldehyde, keto, carboxylic acid ester, carboxylic acid, carbonate, carboxylate, cyano, alkylsilane and alkoxysilane groups and carboxamide groups.

Polythiophenes in which A represents an optionally substituted C₂-C₃-alkylene radical are particularly preferred. Poly(3,4-ethylenedioxythiophene) is very particularly preferred as the polythiophene.

The polythiophenes can be neutral or cationic, wherein cationic polythiophenes are particularly preferred. The term “cationic” only relates to the charges on the polythiophene main chain. The polythiophenes can carry positive and negative charges in the structural unit, depending on the substituent on the radicals R, the positive charges being on the polythiophene main chain and the negative charges optionally being on the radicals R substituted by sulphonate or carboxylate groups. In this context, the positive charges of the polythiophene main chain can be partly or completely satisfied by the anionic groups optionally present on the radicals R. Overall, in these cases the polythiophenes can be cationic, neutral or even anionic. Nevertheless, in the context of one embodiment they are all regarded as cationic polythiophenes, since the positive charges on the polythiophene main chain are the deciding factor. The positive charges are not shown in the formulae, since their precise number and position cannot be determined absolutely. However, the number of positive charges is at least 1 and at most n, where n is the total number of all recurring units (identical or different) within the polythiophene.

For compensation of the positive charge of the cationic polythiophene, it is preferred that conductive polymer further includes a polymeric anion which is in one embodiment based on polymers functionalized with acid groups. Anions of polymeric carboxylic acids, such as polyacrylic acids, polymethacrylic acid or polymaleic acids, or of polymeric sulphonic acids, such as polystyrenesulphonic acids and polyvinylsulphonic acids, are possible in particular as the polyanion. These polycarboxylic and -sulphonic acids can also be copolymers of vinylcarboxylic and vinylsulphonic acids with other polymerizable monomers, such as acrylic acid esters and styrene. Polyanions which are furthermore possible are perfluorinated, colloid-forming polyanions, which are commercially obtainable, for example, under the name Nafion®. The molecular weight of the polymers which are functionalized with acid groups and supply the polyanions is preferably 1,000 to 2,000,000, particularly preferably 2,000 to 500,000. The polymers functionalized with acid groups or their alkali metal salts are commercially obtainable, e.g. polystyrenesulphonic acids and polyacrylic acids, or can be prepared by known processes (see e.g. Houben Weyl, Methoden der organischen Chemie, vol. E 20 Makromolekulare Stoffe, part 2, (1987), p. 1141 et seq.). A particularly preferred polyanion is an anion of polystyrene sulfonic acid.

The conductive polymer preferably is a complex of a cationic polythiophene and a polymeric anion, particularly preferably a PEDOT/PSS-complex. Such complexes are obtainable by polymerizing the thiophene monomers, preferably 3,4-ethylenedioxythiophene, oxidatively in an aqueous solution in the presence of the polymeric anions, preferably by oxidatively polymerizing 3,4-ethylene-dioxythiophene in the presence of an anion of polystyrenesulphonic acid.

### Thermoplastic Polymers

The thermoplastic polymers that are preferably used for the preparation of the bodies according to one embodiment in the 3D-printing process are preferably selected from the group consisting of acrylate or methylmethacrylate polymers or copolymers, acrylonitrile-butadiene-styrene polymers (ABS), polyvinylidene fluoride polymers, polyurethane polymers, polyolefin polymers, polyester polymers, polyamide polymers, polyimide polymers, styrene-ethylene-butylene styrene (SEBS) polymers, polyether ether ketone (PEEK) polymers, polyphenylene sulfide (PPS) polymers, polysulfone polymers, ethylene-vinyl acetate (EVA) polymers, polyethalene-naphthalate (PEN) polymers, aromatic polycarbonate polymers and mixtures of two or more of these polymers.

The term “thermoplastic polyester polymer” refers to those polyester polymers which are thermoplastic. Suitable thermoplastic polyester polymers for use herein may include one or more of the following: polylactic acid (PLA) polymers; polyhydroxyalkanoate (PHA) polymers; or polycaprolactone (PCL) polymers. These thermoplastic polyester polymers may be used in pure form or as a blend with each other, and may include other additives such as plasticizers, fillers, colorants, etc.

The term “polylactic acid or polylactide (PLA) polymers” refers to thermoplastic aliphatic polyesters formed from a lactic acid or a source of lactic acid, for example, renewable resources such as corn starch, sugarcane, etc. The term PLA may refer to all stereoisomeric forms of PLA including L- or D-lactides, and racemic mixtures comprising L- and D-lactides. For example, PLA may include D-polylactic acid, L-polylactic acid (also known as PLLA), D,L-polylactic acid, meso-polylactic acid, as well as any combination of D-polylactic acid, L-polylactic acid, D,L-polylactic acid and meso-polylactic acid. PLA polymers useful herein may have, for example, a number average molecular weight in the range of from about 15,000 to about 300,000 and may have melting points in the range of from about 150° to about 160° C. In preparing PLA polymers, bacterial fermentation may be used to produce lactic acid, which may be oligomerized and then catalytically dimerized to provide the monomer for ring-opening polymerization. PLA polymers may be prepared in a high molecular weight form through ring-opening polymerization of the monomer using, for example, a stannous octanoate catalyst, tin(II) chloride, etc. PLA polymers may be available in a variety of grades that differ in a molecular weight and degree of crystallinity Commercially available PLA polymers suitable for use herein may be obtained, for example, from Nature Works, Inc., such as the Ingeo™ 3, 4, and 6 series of PLA polymers, which includes: 3001D; 3052D; 3100HP; 325 ID; 3260HP; 4032D; 4043D; 4044D; 4060D; 6060D; 6100D; 6201D; 6202D; 6252D; 6260D; 6302D; 6361D; 6362D; 6400D; 6752D; or 10361D.

The term “polyhydroxyalkanoate (PHA) polymers” refers to thermoplastic aliphatic polyesters which may be produced by polymerization of the respective monomer hydroxy aliphatic acids (including dimers of the hydroxy aliphatic acids), by bacterial fermentation of starch, sugars, lipids, etc. PHAs may have melting points in the range of from about 40° to about 180° C. and may include one or more of: poly-beta-hydroxybutyrate (PHB) (also known as poly-3-hydroxy-butyrate); poly-alpha-hydroxybutyrate (also known as poly-2-hydroxybutyrate); poly-3-hydroxypropionate; poly-3-hydroxyvalerate; poly-4-hydroxybutyrate; poly-4-hydroxyvalerate; poly-5-hydroxyvalerate; poly-3-hydroxyhexanoate; poly-4-hydroxyhexanoate; poly-6-hydroxyhexanoate; polyhydroxybutyrate-valerate (PHBV); etc., including copolymers, blends, mixtures, combinations, etc., of different PHA polymers, etc. PHAs may be synthesized by methods disclosed in, for example, U.S. Pat. Nos. 7,267,794, 7,276,361 or 7,208,535.

The term “polycaprolactone (PCL) polymers” refers to thermoplastic aliphatic polyesters which may be prepared by ring opening polymerization of ε-caprolactone using a catalyst such as stannous octoate and may have a melting point about 60° C.

Suitable polymers to be used in the process according to one embodiment include Desmopan 9385 A, Vestamid X7167 nf-pA12, Conductive ABS Nanocyl, Lexan 141R, Cyrolite G20-100, Dryflex UV 902858, Medalist MD-12130H, Desmopan 9370 AU, Grilamid XE4010, Makrolon 2805, Terluran PG35, Lexan 940, Terlux 2802, Meliflex M8260BLT01, GM9450F, Adsyl 5C 30 F, FX381/17, Luvocom 3F 9936, Luvovom 3F 9875, Plexiglas 7N, Lustran ABS 348, MECO-Plast-02-001, 100-GB06, PHA 3444, Marlex HHM 5502BN, PETG-T, PBS, PCU Bionate55D and mixtures thereof.

### The Process According to One Embodiment

The process for the preparation of the bodies according to one embodiment is preferably a 3D-printing process, wherein the body is prepared in layers by depositing droplets of a molten polymer onto the surface of a substrate or—if the layer is not the very first layer—onto the surface of a previously applied layer, wherein in the preparation of at least some of the layers a first area and a second area are formed which preferably differ with respect to the density in which the droplets are deposited.

Such a process can be performed using devices such as the Arburg Freeformer (Arburg GmbH+Co. KG, LoBburg, Germany). This is a device for the production of a three-dimensional objects made of a solidifiable material (which is either present in a fluid phase in its initial state or can be fluidified) by a sequential discharge of drops as disclosed in US 2012/156319 A1. The device includes
at least one construction space for constructing the object,
at least one processing unit for processing of the solidifiable material into the fluid phase,
at least one material reservoir for the fluid phase with at least one discharge unit for the sequential discharge of the solidifiable material in the form of discrete drops in direction of the construction space through a discharge orifice furnished with a synchronizable closing mechanism,
at least one pressure generating unit for generating pressure on the fluid phase in the material reservoir,
   wherein the closing mechanism has an elastic deformable solid body joint.

The Arburg Freeformer is characterized by a piezo nozzle cap that opens and closes up to a hundred times per second to produce tiny droplets of thermoplastic material. The Freeformer builds up the three-dimensional object layer by layer from the droplets. The desired volume of the drop preferably lies in the range from 0.01 to 0.5 mm³, preferably in the range of 0.05 to 0.3 mm³ and in particular preferably in the region of about 0.1 mm³. The diameter of the discharge opening is preferably smaller or equal to 1 mm, more preferably about 0.1 mm. The layer thickness results from the respective droplet height and usually varies between 0.14 and 0.34 mm, also depending on the nozzle that is used. The shape of the discharged droplets is also influenced by the viscosity of the material.

Printers such as the Arburg Freeformer 200-3X or 300-3X not only allow the use of two or more different thermoplastic materials for the formation of a three-dimensional object, they are also able to deposit these different materials with different filling factors when depositing the droplets of the molten materials. It is thus possible to prepare three-dimensional objects having areas with a different porosity (and which additionally may include polymers with a different compression modulus) which, depending on their porosity, may be selectively impregnated with conductive materials in order to increase the conductivity in certain areas or the three-dimensional object in a targeted way.

In a 3D-printing process that is based on additive layer manufacturing (ALM), such as the printing process that is performed when using the Arburg Freeformer, process, the 3D-body is usually formed layer-up-layer, each layer being formed line-next to-line in a given layer and each line being formed droplet-next to droplet in a given line. To adjust the porosity of a given segment in a 3D-printing process by means of 3D-printers such as the Arburg Freeformer
i) the distance in which two subsequent droplets in a given line are deposited next to each other onto a substrate or onto a layer that has been previously printed can be varied (for example by adjusting the frequency in which piezo nozzle cap in an Arburg Freeformer opens and closes),
ii) the distance in which two consecutive print lines are printed in a given layer can be varied (by an appropriate positioning of the printing nozzle relative to the substrate onto which the drops are to be deposited), or
iii) measures i) and ii) can be combined.

Furthermore, when forming a given layer of a 3D-object by means of the above described additive layer manufacturing (ALM) process it may also be advantageous to additional print a supporting polymer in the outer area of each layer (which in the finished 3D-object than covers the outer surface of the body or at least a part of it) in order to provide a sufficient stability of the 3D-object and to improve the form stability during the printing process. In this context thermoplastic polymers such as Armat 21 (Arburg, Germany) are preferred which later can easily be removed by means of alkaline aqueous solutions.

According to a first preferred embodiment of the process according to one embodiment only one thermoplastic material is used for the formation of both, the first and the further segment, wherein for the formation of the first and the further segment this thermoplastic material is deposited in different filling factors, which in turn leads to a different porosity of the first and the further segment. To obtain the desired difference in the electrical conductivity of the first and the further segment, one of these segments is—after the formation of the body—brought into contact with a conductive material. This can be accomplished by selectively coating the outer surfaces of the first or the further segment with a conductive material, by impregnating the first or the further segment with a conductive material (such as a PEDOT/PSS-dispersion) or by a combination of these approaches. The thermoplastic material that is used for the formation of the first and the further segment can be a non-conductive thermoplastic polymer or a conductive material that includes a non-conductive thermoplastic polymer and a conductive component. If a conductive material is used for the formation of the first and the further segment, both segments are characterized by a certain electrical conductivity and the conductivity in one of these segments can than be further improved by one of the approaches described before (i. e. by coating the outer surface of one of the segments with a further conductive material or by impregnating one of these segments with a conductive material).

According to a second preferred embodiment of the process according to one embodiment two different thermoplastic materials are used for the formation of the first and the further segment, wherein for the formation of the first and the further segment these thermoplastic materials are also deposited in different filling factors, which in turn leads to a first and a further segment which differ in both, the material from which they have been formed and the porosity. Thus, for the formation of the first polymer segment a first thermoplastic material can be used (which may be a conductive material having a first conductivity or a non-conductive material) and for the formation of the further segment a second thermoplastic material can be used (which again may also be a conductive material having a second conductivity or a non-conductive material). If the two materials differ with respect to their electrical conductivity, the process directly leads to the formation of a body comprising two segments which differ with respect to both, their porosity and their electrical conductivity. If two non-conductive materials are used, the conductivity in one of these segments can than be further improved by one of the approaches described before (i. e. by coating the outer surface of one of the segments with a further conductive material or by impregnating one of these segments with a conductive material).

For the formation of the first and the further segment in the process according to one embodiment, particularly in the process according to the first and the second embodiment as described above,
the first segment is prepared using a fill factor f1 in the range from 5 to 100%, preferably in the range from 10 to 90%, more preferably in the range from 15 to 80% and even more preferably in the range from 25 to 70%;
the further segment is prepared using a fill factor f1 in the range from 5 to 100%, preferably in the range from 10 to 90%, more preferably in the range from 15 to 80% and even more preferably in the range from 20 to 70%.

Furthermore, it is also preferred that for the formation of the first and the further segment in the process according to one embodiment, particularly in the process according to the first and the second embodiment as described above, the following condition is fulfilled:
f2>1.25×f1,
more preferably f2>1.5×f1,
even more preferably f2>1.75×f1,
even more preferably f2>2×f1,
even more preferably f2>4×f1.

### The Therapeutic Device According to One Embodiment

The therapeutic current delivery or current receiving device can be any type of monitoring or stimulation device that is known to the person skilled in the art, such as, for example, an electrograph (ECG) device, an electroencephalograph device, an electromyography device, a transcutaneous electrical nerve stimulation device, an electrical muscle stimulation device, a neuromuscular stimulation devices, or a functional electrical stimulation device.

### Test Methods

The following test methods are used in one embodiment. In absence of a test method, the ISO test method for the feature to be measured being closest to the earliest filing date of the present application applies. In absence of distinct measuring conditions, standard ambient temperature and pressure (SATP) as a temperature of 298.15 K (25° C., 77° F.) and an absolute pressure of 100 kPa (14.504 psi, 0.986 atm) apply.

To determine the porosity, the electrical conductivity and the compression modulus in the different segments of the body according to one embodiment the test methods described below can be used. If the size of the segments is to small or if the segments in the body are not accessible to determine these parameters, it is recommended to prepare these segments in individual form and in sizes that are required for the specimens used in these test methods, wherein specimens of the segments are printed by 3D-printing under the same conditions (fill factor, temperature and viscosity of the molten polymer etc.) as they have been printed when preparing the body according to one embodiment or when applying the process according to one embodiment.

### Porosity

The porosity of can determined one the basis of the geometrical density with the following steps:
1) Dry the sample for at least 2 h at 85° C.
2) Measure the exact geometry of the sample, preferably contact less as with a Mitutoyo S200Z, and calculate the volume V.
3) Measure the mass m of the sample.
4) Calculate the bulk density ρ_{B}=M/V.
5) Calculate porosity P in % with the theoretical density ρₜₕₑₒ of the material as P=(ρₜₕₑₒ−ρ_{B})/ρₜₕₑₒ 100%.

### Electrical Conductivity

The electrical conductivity is determined according to ASTM B193 (Standard Test Method for Resistivity of Electrical Conductor Materials) with the following steps:
1) A bar of 30 mm length l of the sample is provided having a uniform square cross-section A of 5 mm edge length.
2) The dimensions are determined using contact-less measurement such as measurement projector Mitutoyo QS200Z.
3) The resistance R is measured.
4) The conductivity is calculated as σ=1/p=1/(A×R).

### Compression Modulus

The compression modulus of the first and the further segment of the body according to one embodiment can be determined on the basis of DIN EN ISO 3386-2, with the following steps:
1) Three samples of each segment were measured whereas each sample is a cylinder with a thickness of 15 mm and a diameter of 5 mm whereas the z-direction of the sample (that is, along the thickness) along which the force will be applied coincides with the z-direction during printing.
2) Measurement is conducted at least 72 h after production of the samples and after at least 24 h of conditioning the undeflected, undistorted samples at 23° C. at 50% relative humidity.
3) Compression is performed with 100 mm/min until a compression of 70% of the initial test piece thickness is achieved and then decompression to the initial test piece thickness is performed.
4) The procedure is repeated immediately three times while the fourth compression cycle is recorded for evaluation.
5) The slope of the initial linear section in the compression-strain curve is the Young's modulus Y from which the compression modulus K is calculated as

K=Y/(3-6v) with the Poisson ratio v approximated with 0.3.

### EXAMPLES

Embodiment are now explained in more detail by examples and drawings given by way of example which do not limit them.

### Example 1

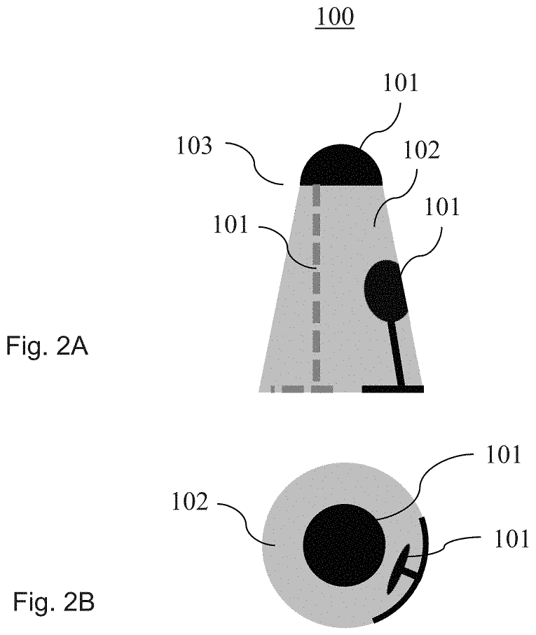

A three-dimensional body as shown in FIG. 2A that can later be used as an in-ear multiple electrode has been printed in an Arburg Freeformer 300-3X using the following conditions and parameters:

| | mass | | | |
|---|---|---|---|---|
| | preparation 1 | mass | mass | |
| | ABS | preparation 2 | preparation 3 | |
| | (Terluran | Armat 21 | Thermoflex | |
| material | GP35 300-3X) | 300-3X | EC 80.1 | unit |
| nozzle diameter | 0.2 | 0.2 | 0.2 | mm |
| (built in) | | | | |
| layer thickness | 0.2 | | | mm |
| number of layers | 124 | | | |
| droplet closing | 367 | 487 | 565 | ×10³ |
| factor | | | | |
| number of | 100 | 105 | 565 | ×10³ |
| droplets | | | | |
| material feed | 35 | 45 | 35 | ° C. |
| temperature | | | | |
| temperature | 190 | 210 | 160 | ° C. |
| zone 1 | | | | |
| temperature | 220 | 235 | 190 | ° C. |
| zone 2 | | | | |
| temperature | 240 | 220 | 210 | ° C. |
| deposition zone | | | | |
| temperature | 90 | | | ° C. |
| construction | | | | |
| space | | | | |
| lowering | 100 | 100 | 100 | ° C. |
| temperature | | | | |
| zone 1 | | | | |
| lowering | 90 | | | ° C. |
| temperature | | | | |
| construction zone | | | | |
| melt cushion | 1.5 | 1.5 | 1.5 | mm |
| circumferential | 4 | 4 | 3 | m/min |
| speed | | | | |
| dynamic pressure | 50 | 40 | 40 | bar |
| metering stroke | 10 | 10 | 6 | mm |

The ABS-polymer forms the “further segment” of the body according to one embodiment and constituted the matrix polymer into which a conductive segment that is formed from the PTS-polymer (i. e. the “first segment” of the body according to one embodiment) is embedded and on the surface of which such a conductive segment is localized.

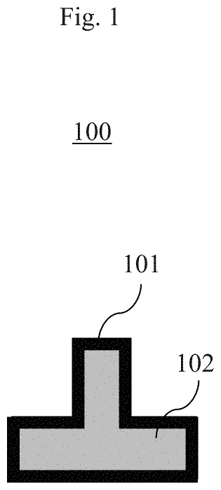

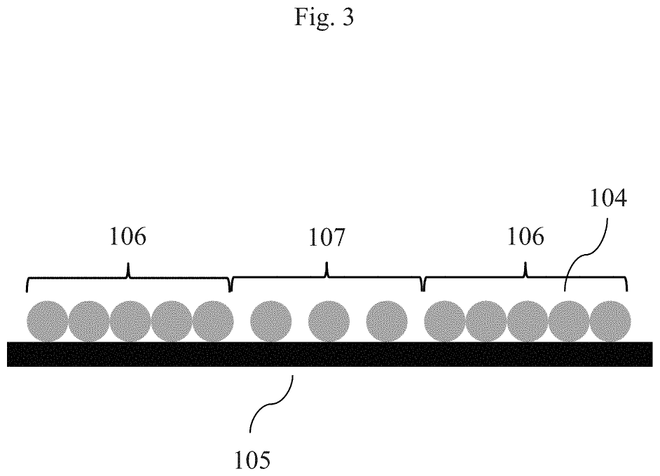

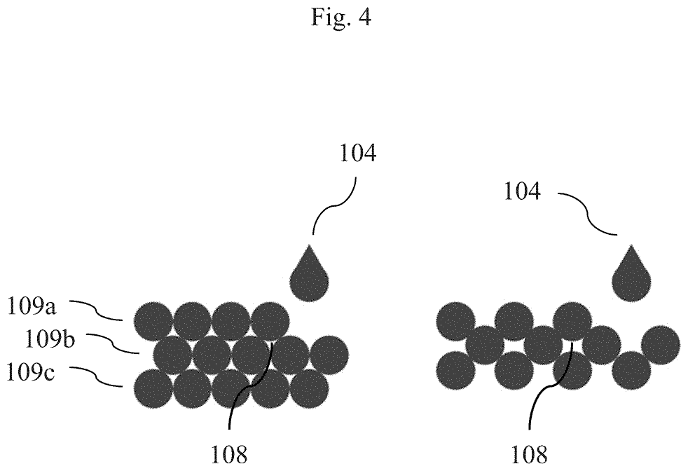

FIG. 1 is a cross-sectional scheme of the body 100 according to one embodiment, wherein the body 100 includes at least one first segment 101 and at least one further segment 102, wherein these segments differ in both, the porosity and the electrical conductivity. The first and the further segment 101,102 are in seamless contact with each other. When producing the body 100 according to one embodiment by means of a 3D-printing in the border area between these two segments droplets 104 (see FIGS. 3 and 4) of the molten thermoplastic material used to prepare the first segment 101 and droplets 104 (see FIGS. 3 and 4) of the molten thermoplastic material used to prepare the further segment 102 flow into each (i. e. they form a coherent material upon solidification of the molten thermoplastic material).

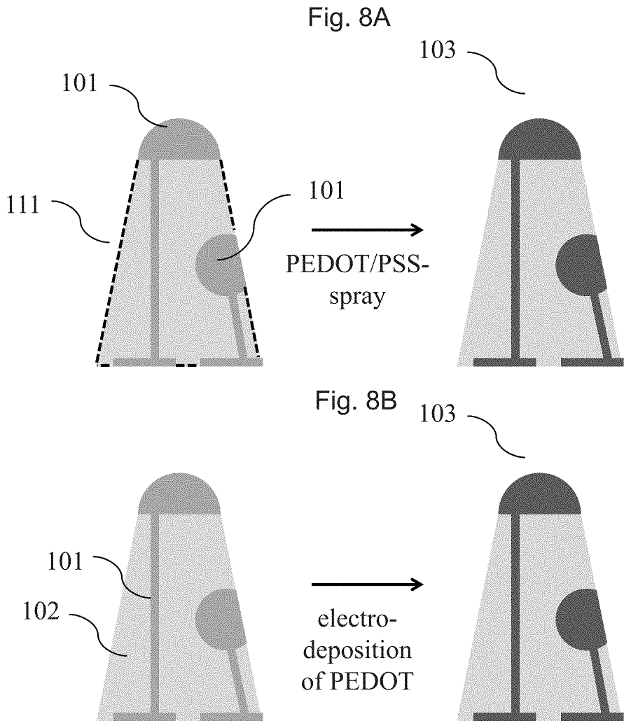

FIG. 2A is a cross-sectional scheme of the body 100 according to one embodiment in the design of an in-ear multiple electrode 103. FIG. 2B shows the same the in-ear multiple electrode 103 in a top view. The multiple electrode 103 includes first segments 101 forming the electrodes and a further segment 102 serving as the base in which the electrodes 101 are embedded (see the pathways 101 on the left that are shaded in dark grey) or onto they have been deposited (see the pathways 101 on the top and on the right that are shaded in black and that in one embodiment serve for direct skin contact). As shown in FIG. 2A, the embedded pathway 101 contacts the electrode 101 on the top of the multiple electrode 103 and thus allows a connection of that electrode 101 with a measuring device (not shown in FIG. 2A). In one embodiment, in such an in-ear multiple electrode 103 the base 102 is made from a soft and deformable material, whereas the electrodes are manufactured from a harder material. When producing the in-ear multiple electrode 103 by means of a 3D-printing process the thermoplastic material used to prepare the base 102 is preferably a non-conductive thermoplastic material, which upon solidification forms a soft material, whereas the electrodes 101 are either formed from a conductive thermoplastic material or from a non-conductive thermoplastic material which, after the formation of the multiple electrode 103 is brought into contact with a conductive material in order to provide the required conductivity (as also shown in FIG. 8).

FIG. 3 is a scheme illustrating the deposition of droplets 104 onto the surface of a substrate 105 in a 3D-printing process according to one embodiment. As shown in FIG. 3 two different areas 106 and 107 are formed in which the droplets are deposited in a different density. In a three-dimensional structure in which by means of such as process several successive layers 109, 109a, 109b, 109c are produced (see FIGS. 4, 5 and 7), polymer bodies 100 are obtained comprising two or more different segments 101, 102 which differ with respect to their porosity. If, in addition to the different density in which the droplets 104 are deposited, also different thermoplastic materials are used, bodies 100 can be obtained in which the segments 101, 102 not only differ with respect to their porosity, but also with respect to their compression modulus (i. e. their softness). In order to obtain the desired difference in the electrical conductivity of the segments 101, 102 it is possible to either use thermoplastic materials which differ with respect to their electrical conductivity and/or to bring one of these segments into contact with a conductive material (or to combined these measures). In one embodiment, the segment with the higher porosity is impregnated with a conductive material such as a conductive polymer or a conductive polymer is formed within the pores 108 of the segment with the higher porosity.

FIG. 4 shows two further schemes illustrating the deposition of droplets 109 in a 3D-printing process according to one embodiment, in which three successive sheet layers 109a, 109b and 109c are shown. As can be seen from that figure, pores 108 are formed the size of which can be adjusted by the printing density that is used. In a subsequent process step a conductive material can be introduced into these pores 108 in order to increase the electrical conductivity in selected areas of the body 100.

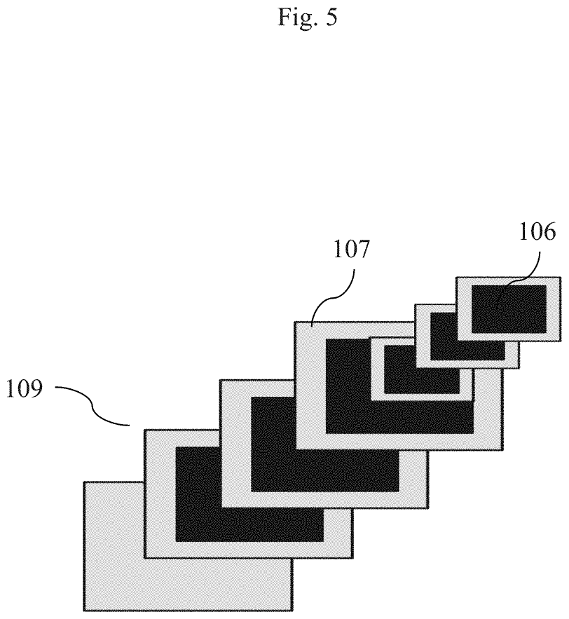

FIG. 5 illustrates the formation of the body 100 shown in FIG. 1 by means of the process according to one embodiment. As can be seen, layers 109 which include areas 106 (grey), 107 (black) in which the droplets of the thermoplastic polymer have been deposited in a different density (as shown in FIGS. 3 and 4) are formed one after another, thereby obtaining the polymer body with a structure as shown in FIG. 1 (not all the layers that are necessary to form the electrode of FIG. 1 are shown). The first polymer segment 101 that is obtained by the successive deposition of the areas 107 is characterized by a higher porosity, compared to the second polymer segment 102 that is obtained by the successive deposition of the areas 106.

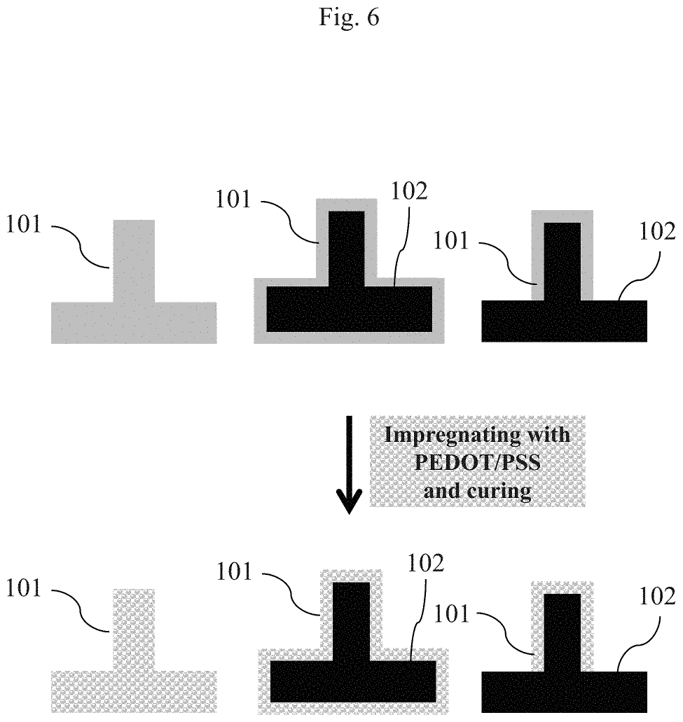

FIG. 6 is a scheme showing the process step of impregnating certain segments of the body according to one embodiment shown in FIG. 1 with PEDOT/PSS. The body 100 in the centre of FIG. 6 corresponds to the one shown in FIG. 1 and obtained by the process shown in FIG. 5. The body on the left is not according to one embodiment whereas the body 100 on the right is a slight modification of the body 100 in the centre. The bodies 100 in the centre and on the right include a first segment 101 in the outer region that is characterized by a high porosity and a further region 102 serving as the base of the body 100. To provide the more porous segment 101 with the desired electrical conductivity, the bodies are impregnated with a conductive material, such as a PEDOT/PSS-dispersion. Suitable dispersions that can be used for that purpose are formulation such as Tecticoat™ (Heraeus, Germany). In the alternative, it is also possible to impregnate the more porous regions with compositions comprising monomers of a conductive polymer and to polymerize these monomers in situ for the formation of a conductive polymer by means of electrodeposition. Compositions suitable for that purpose are formulation such as Amplicoat™ (Heraeus, Germany). Furthermore, the more porous region can also be impregnated with conductive inks based on metal particles or metal-organic complexes. To limit the impregnation of the more porous regions with conductive materials to well defined areas, it is also advantageous to apply a protective coating 111 onto those areas that are not intended to be impregnated (as shown in FIG. 8A).

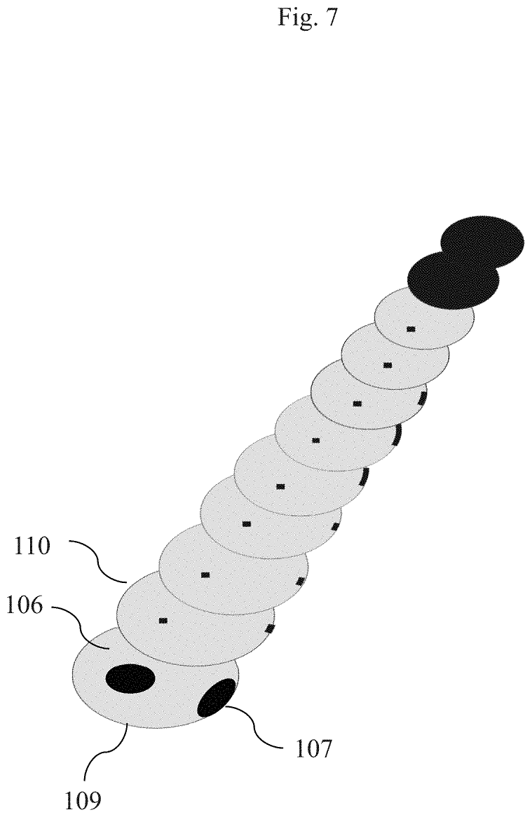

FIG. 7 shows the formation of the in-ear multiple electrode 103 that is illustrated in FIG. 2A by means of the 3D-printing process according to one embodiment. As can be seen, the in-ear multiple electrode 103 is formed layer-by-layer (again not all the layers that are necessary to form such an electrode are shown), wherein at least some of the layers 109 include areas 106,107 in which the drops 104 of the thermoplastic material have been deposited in a different density (as shown in FIGS. 3 and 4). The process shown in FIG. 7 leads to a body 100 with the structure of the in-ear multiple electrode shown in FIG. 2A which includes a first segment 101 serving for the formation of the electrodes and a further segment 102 serving as the base in which the electrodes 101 are embedded.

FIGS. 8A and 8B show a scheme illustrating the process step of introducing PEDOT in certain segments of the in-ear multiple electrode 103 that has been obtained by the process shown in FIG. 7. According to the approach shown in FIG. 8a the outer surface of the multiple electrode 103 is partially covered with a polymer coating 110. The areas of the first segment 101, however, remain uncoated. In order to increase the electrical conductivity in the first segment 101, PEDOT/PSS, for example in form of the formulation Tecticoat™, is sprayed upon these surfaces. Upon drying and removal of the protective polymer coating 111 an in-ear multiple electrode 103 as shown in FIG. 2a is obtained in which the PEDOT/PSS-impregnated areas form the electrodes. According to the approach shown in FIG. 8b the multiple electrode 103 that has been obtained by the process shown in FIG. 7 is impregnated with a composition comprising monomers of a conductive polymer and the monomers are subsequently polymerized in situ for the formation of a conductive polymer by means of electrodeposition. Compositions suitable for that purpose are formulation such as Amplicoat™ (Heraeus, Germany). As can be seen in FIG. 8B, this process is particularly suitable to also increase the electrical conductivity in segments of the body 100 which are completely embedded in the base (see segment with reference number 102 in FIG. 8B).

Although specific embodiments have been illustrated and described herein, it will be appreciated by those of ordinary skill in the art that a variety of alternate and/or equivalent implementations may be substituted for the specific embodiments illustrated and described without departing from the scope of the present embodiments. This application is intended to cover any adaptations or variations of the specific embodiments discussed herein. Therefore, it is intended that these embodiments be limited only by the claims and the equivalents thereof.

1. A body, comprising
i) at least one first polymer segment having a porosity p1 and an electrical conductivity c1,
and
ii) at least one further polymer segment being in seamless contact with the at least one first polymer segment and having a porosity p2≠p1 and an electrical conductivity c2≠c1.

2. The body according to claim 1, wherein
p1>p2, and
c1>c2
or
p1>p2, and
c2>c1.

3. The body according to claim 1, wherein the first polymer segment has an electrical conductivity c1 of at least 0.001 S/cm and wherein the further polymer segment has an electrical conductivity c2 of less than 1×10⁻⁹ S/cm.

4. The body according to claim 1, wherein at least one segment selected from the first polymer segment and the further polymer segment is made from a conductive material that comprises a non-conductive polymer matrix and a conductive component dispersed therein.

5. The body according to claim 1, wherein at least one segment selected from the first polymer segment and the further polymer segment is made from a non-conductive material.

6. The body according to claim 1, wherein the body is comprised in a device selected from the group consisting of, or is a device selected from the group consisting of, a biosensor, a body-worn device having electrically sensing functionality and a body-worn device selected from the group consisting of a headphone, glasses, a headband, a chest-strap, an armband, a patch, a garment or a wrist-worn device.

7. A process for the preparation of a body, comprising
I) preparing, by means of 3D-printing, at least one first polymer segment having a porosity p1, by using a first fill factor f1, and
II) preparing, by means of 3D-printing, at least one further polymer segment being in seamless contact with the at least one first polymer segment having a porosity p2≠p1, by using a second fill factor f2≠f1;
III) optionally bringing into contact a conductive material with a segment selected from the first polymer segment and the further polymer segment.

8. The process according to claim 7, wherein the body is prepared in layers by depositing droplets of a molten polymer onto a surface of a substrate or onto a surface of a previously applied layer, onto the outer surface of a previously applied layer, wherein in the preparation of at least some of the layers a first area and a second area are formed which differ with respect to the density in which the droplets are deposited.

9. The process according to claim 7, wherein at least one segment selected from the first polymer segment and the further polymer segment is made from a conductive material that comprises a non-conductive polymer matrix and a conductive component dispersed therein.

10. The process according to claim 7, wherein at least one segment selected from the first polymer segment and the further polymer segment is made from a non-conductive material.

11. The process according to claim 7, wherein at least one segment selected from the first polymer segment and the further polymer segment comprises void spaces and wherein in process step III) a conductive component is introduced into at least some of the void spaces or wherein in process step III) at least a part of the surface of at least one segment selected from the first polymer segment and the further polymer segment is coated with a conductive component.

12. A body, obtainable by the process according to claim 7.

13. The body according to claim 1, wherein the body is comprised in a device selected from the group consisting of, or is a device selected from the group consisting of, a biosensor, a body-warn device having electrically sensing functionality and a body-worn device selected from the group consisting of a headphone, glasses, a headband, a chest-strap, an armband, a patch, a garment or a wrist-warn device.

14. Use of the body according to claim 1 for electrophysical measurements, wherein at least a part of at least one segment selected from the first polymer segment and the further polymer segment comes into direct contact with the skin.

15. A therapeutic current delivery or current receiving system comprising:
a) a therapeutic current delivery or current receiving device;
b) at least one cable connector connected to the therapeutic current delivery or current receiving device;
c) at least one body according to claim 1 connected to the at least one cable connector.
